(19) United States
(12) Patent Application Publication
(10) Pub. No.: US 2017/0340849 A1
(43) Pub. Date: Nov. 30, 2017

(54) Non stretch, loop fastening NEO-prene

(71) Applicant: Beverley Hilda Anderson, Canberra (AU)

(72) Inventor: Beverley Hilda Anderson, Canberra (AU)

(21) Appl. No.: 15/526,737

(22) PCT Filed: Nov. 13, 2015
(86) PCT No.: PCT/AU2015/050713

§ 371 Date: May 12, 2017

(30) Foreign Application Priority Data
Nov. 18, 2014 (AU) PCT/AU2014/101151

(51) Int. Cl. A61M 16/06; A61M 16/08

(52) U.S. Cl. CPC A61M 16/0605; A61M 16/0816; A61M 16/0694; A61M 2240/00; A61M 2205/42

(57) ABSTRACT

The Non stretch, loop fastening NEO-prene, used in my various products, for our sick & premature neonate's, allows more effective & secure CPAP (continuous positive airway pressure) therapy. It also decreases the risks of pressure injury like skin pressure areas, burns, nasal septal damage & deformation of the preterm head (“Prem Head”). It helps prevent loss of CPAP air pressure (“pop off”), as well as decrease retinal damage of the eyes, & inaccurate oxygen saturation readings leading to poor oxygen management in neonates. It also contributes to developmental care of the neonate by limiting damaging loud noises & bright lights, which have detrimental neurological consequences for neonatal development.

### TECHNICAL FIELD

The non stretch, loop fastening NEO-prene was developed to complement & improve other innovations that I have developed to help nurse sick & premature babies in Neonatal Intensive Care Units & Special Care Nurseries.

### BACKGROUND INFORMATION AND PRIOR ART

I am a Neonatal Intensive Care Nurse, I previously developed the The Cpap Cap System (Registered Design AU147007S) & accessories for the system. Including a chin strap, alternate CPAP anchoring accessories, phototherapy eye protectors & a saturation probe securing device (Sat Wrap). I was not happy with the material that I used with all of these innovations, as it caused pressure areas on the neonates. It was ‘Veltex®’, stitched to ‘Magic Stop’ (non slip matting). I searched for a suitable, alternative material, to no avail. It had to be soft for comfort, ‘non slip’ to keep in place, have a loop fastening surface & most importantly be non stretch. As constriction can cause extensive damage, via pressure, to the fragile preterm neonates. I found neoprene to be the material of choice except that it was stretchy, & when applied wrongly could be catastrophic for the neonate.

### DESCRIPTION OF YOUR INVENTION

So, I laminated 1.5 & 2 mm neoprene sheet with a durable, non stretch, high tear strength, loop fabric. The material, NEO-prene, is then used in all neonatal products which form the Cpap Cap System, as detailed in the drawings. It works exceptionally well. It stays in place, is extremely comfortable for the neonates, causes no pressure related injuries & contributes positively to the neonates developmental care. Ultimately, resulting in a better outcome for the sick neonates.

### DESCRIPTION OF HOW YOUR INVENTION ADDRESSES A TECHNICAL PROBLEM

On the cap: The NEO-prene surround (or rim) keeps the CPAP firmly in place, allowing better oxygenation, via cpap that stays in situ & promotes minimal handling; as well as providing multiple fixation sites to get the CPAP in the idea position; while not causing pressure injury. The NEO-prene also preserves the parietal eminences by padding the dip of the temporal bone & distributes the weight of the head normally placed on the parietal eminences, when the neonate is nursed on their sides. Therefore, helping prevent a flat ‘prem head’. The NEO-prene surround, covers the ears which provides noise reduction for the neonate. Therefore, contributing to a better developmental environment.

On the chin strap & anchors: The NEO-prene chin strap gently but firmly keeps the mouth closed to prevent ‘pop off’ of air pressure through the mouth. The anchors maintain the CPAP device firmly but gently in the neonates nares, increasing oxygenation & decreasing nasal septal damage & necrosis.

Phototherapy eye protector: The NEO-prene blacks out any lux from the phototherapy to prevent retinal damage. It also blacks out ambient light which contributes to a better developmental environment, for preterm neonates.

Sat Wrap: The NEO-prene is used to secure a saturation probe in place, usually on the foot or hand of the neonate. It is non conforming, which allows air to circulate around the probe, decreasing the risk of burns. Being non elastic it avoids constriction which allows better circulation to probe, for more accurate readings & decreases the risk of burns & skin pressure injury. The blackout property of the material stops ambient, as well as, light from phototherapy & heat lamps to interfere with accurate probe readings therefore allowing for better oxygen management.

### LIST OF FIGURES:

FIG. 1:

1 is the dark material is 1.5 or 2 mm neoprene

2 is the white material is a durable, non stretch, high tear strength, loop fabric

FIG. 2:

3 is the lamination of the neoprene & the loop fabric to make NEO-prene. They are laminated with hot melt adhesive.

FIG. 3:

NEO-prene used for the chin strap. Various sizes for different sizes of premature babies, all with an appropriate sized slit to accommodate baby's chin. 4 is the dark drawing is the neoprene side & 5 is the white side is the loop side.

FIG. 4:

NEO-prene used in the construction of the Cpap device anchors. 6 is the loop side of anchor & 7 is the ribbon & ring which is sewn onto the NEO-prene strip to fit onto the nasal portion of the Cpap device, to hold the device insitu.

8 is the neoprene side of the Cpap device anchors

FIG. 5:

NEO-prene used in the construction of the phototherapy/developmental eye protectors. 9 is the neoprene side of the eye protectors & 10 is the loop side of the eye protectors.

FIG. 6:

NEO-prene used in the construction of the Cpap Cap. 11 is the cotton polo material used in the body of the cap, 12 is the neoprene side of the rim sewn onto the edge of the inside of the cap & 13 the double sided loop & hook fastener sewn onto one end of the NEO-prene rim. To securing the cap, at the appropriate place, once it has been placed around the circumference of the baby's head. 14 is the loop fabric side of the rim sewn onto the edge of the outside of the cap.

FIG. 7:

NEO-prene used in construction of the Saturation monitor probe securing wrap (Sat Wrap). 22 is the Neoprene side (inside) of Sat Wrap. 23 is the double sided loop & hook fastener sewn onto wrap to allow adjustable sizing & securing around saturation probes placed on baby's limbs. 24 is the loop fabric side (outside) of Sat Wrap.

FIG. 8:

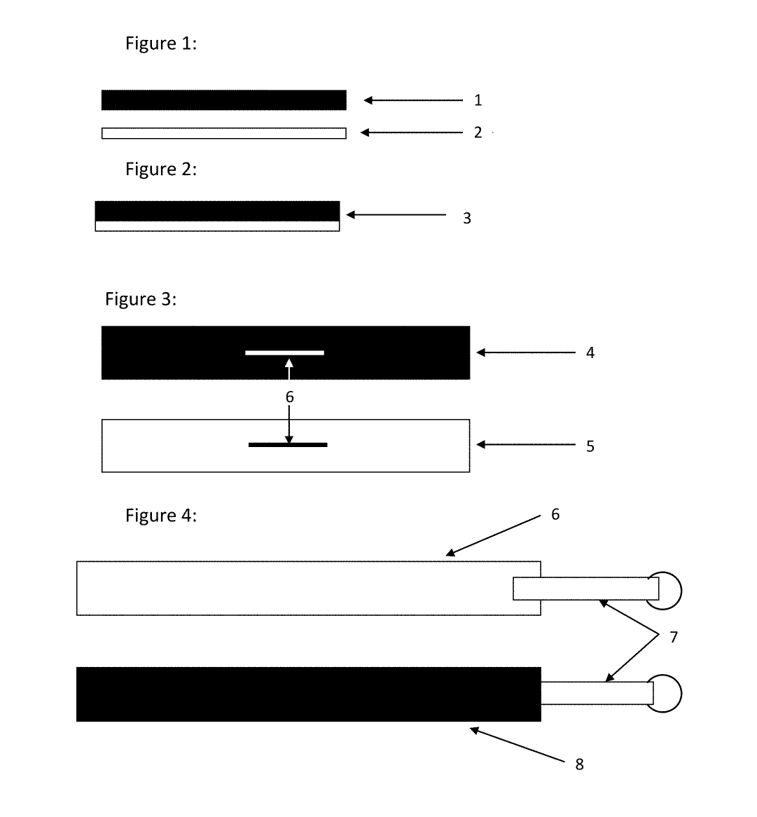

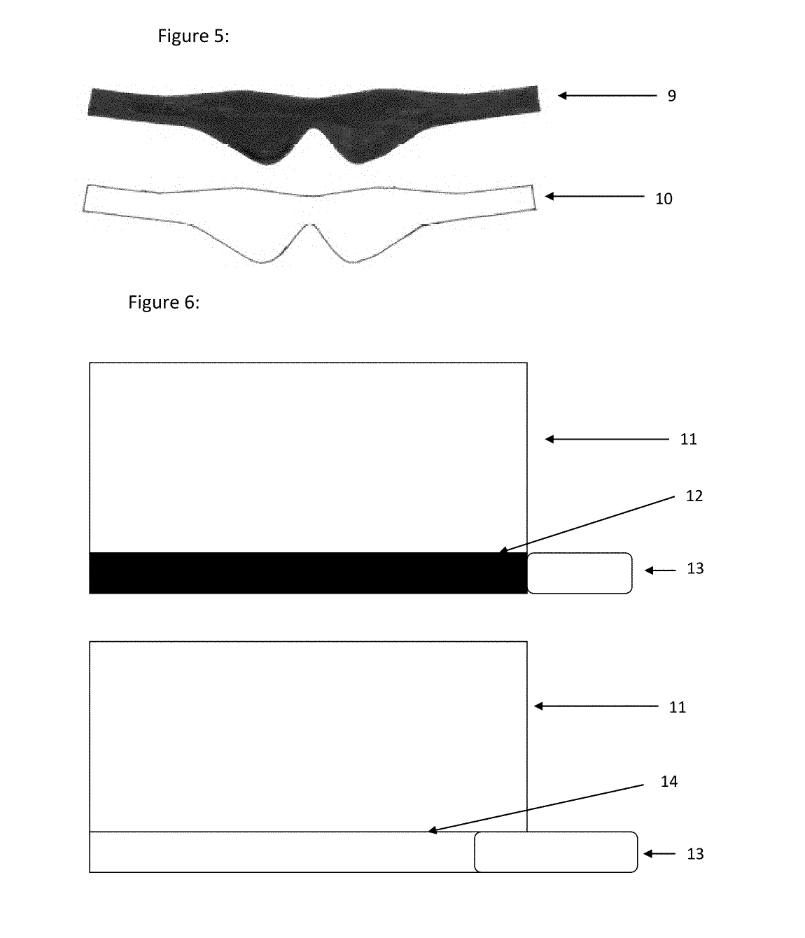

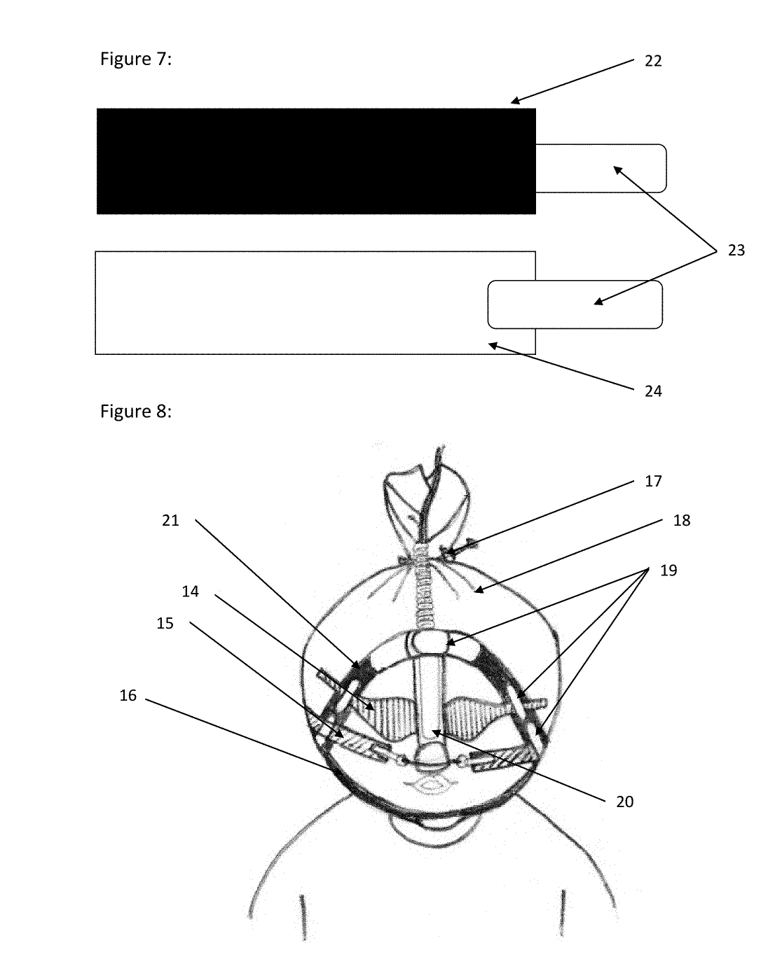

The Cpap Cap System insitu (front view) including Cap, Anchors, Fasteners, Chin Strap & Eye Protectors. 14 is the NEO-prene eye protectors, 15 is the NEO-prene Cpap device anchors, 16 is the NEO-prene chin strap, is the Shockcord tightener which secure the top of the cap & is adjustable to allow for different types of heads. 18 is the cotton polo material making up the body of the cap. 19 are the double sided loop & hook fasteners of different lengths used to secure Cpap device, eye protectors, anchors & chin strap. 20 is the Cpap device. 21 is the NEO-prene rim, used to secure Cpap device, anchors, chin strap & eye protectors to cap via various fasteners.

FIG. 9:

The Cpap Cap System insitu (side view) including Cap, Anchors, Fasteners, Chin Strap & Eye

Protectors. Refer to above number references,

FIG. 10:

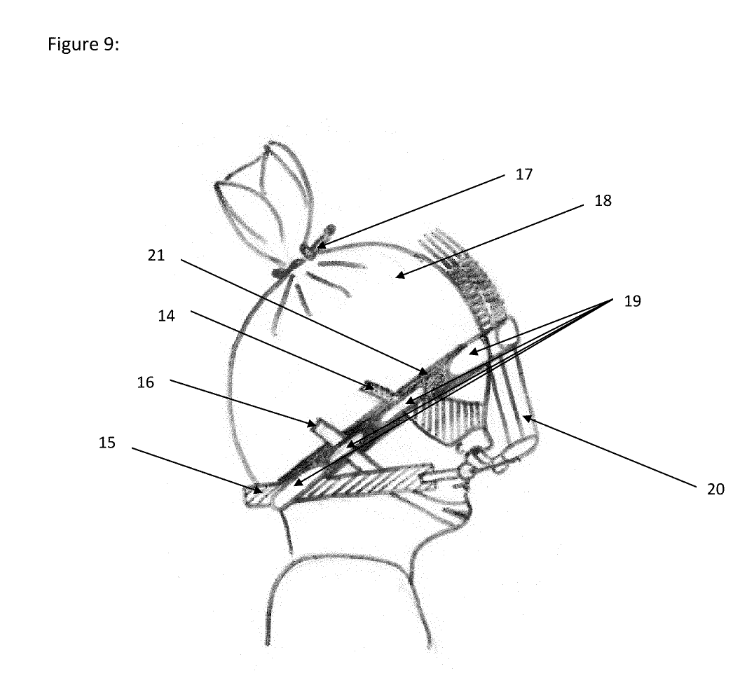

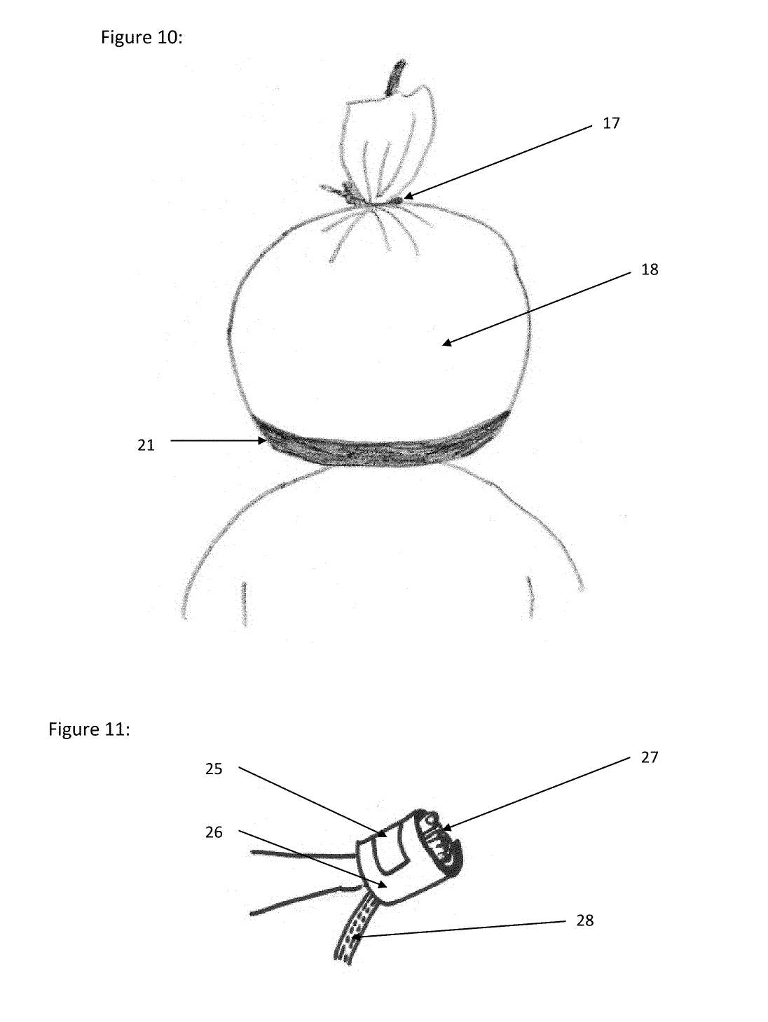

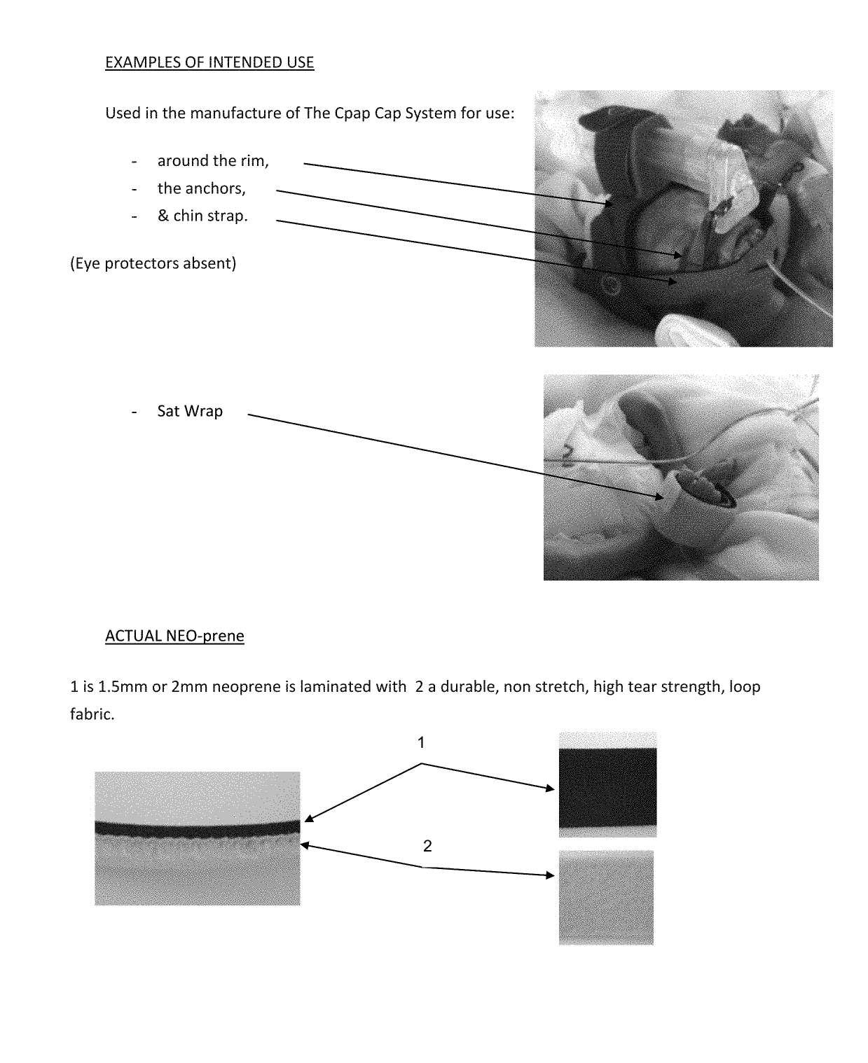

The Cpap Cap System insitu (back view) including Cap, Anchors, Fasteners, Chin Strap & Eye Protectors. Refer to previous number references in FIG. 8.

FIG. 11:

NEO-prene used in construction of the Sat Wrap. 25 is the double sided loop & hook fastener sewn onto the NEO-prene body of the Sat Wrap, to allow for multiple adjustment for different sized baby's feet & hands. 26 is the NEO-prene body of the Sat Wrap. 27 are the baby's toes. 28 is the Saturation probe lead, which is plugged into the Saturation Monitor to record oxygen saturations

What is claimed is:

1) a material used around the rim of the Cpap Cap System to securing a nasally inserted neonatal respiratory Cpap device, via double sided loop/hoop fasteners & anchors also made of the said material. The said material rim can accommodate different sized heads as it is open ended with a double sided loop/hook fastener. The said material also allows for multiple attachment sites for a chin strap & eye protectors, also made of the said material.
The said material allows the respiratory device to be positioned securely within the nares of the neonate; provides comfort for the neonate (no pressure areas or constriction); decreases noise to the neonate as it occludes the ears (good developmental care); & keeps an even pressure around the head & cushions the temporal dip to stop flattening of the parietal eminences which causes “Prem Head”.

2) a material used on the anchor, used with the Cpap Cap System, which has a doubled over piece of ribbon, with a metal ring, sewn to one end. Two anchors (one on either side of face) secure the nasal piece of the Cpap device in the neonate's nares via the ring & ribbon. The said material is then secured to the said material rim of the Cpap cap by double sided loop/hook. fasteners.
The said material maintains the Cpap device firmly but gently in the neonates nares, increasing oxygenation & decreasing nasal septal damage & necrosis.

3) a material used to make a chin strap used with the Cpap Cap System. The said material has a slit in the middle to allow the neonate's chin to poke through to hold the mouth shut. This stops air pressure escaping via the mouth, therefore maintaining CPAP. The chin strap is secured via double sided loop/hoop fasteners attached to the said material on the rim of the Cpap cap.
The said material gently but firmly keeps the mouth closed to prevent loss of the Cpap pressure.

4) a material used to make eye protectors, used with the Cpap Cap System. They are attached to the said material rim of the Cpap cap by double sided loop/hook fasteners.
The said material allows the neonate to have phototherapy without the risk of retinal damage & allows for better developmental care by also blocking out bright lights.

5) a material used to make Sat Wraps, used to secure a Pulse Oximeter probe to a neonates extremities.
The said material allows the neonate to have their oxygen saturation monitored without the risk of pressure injury & burns; & also allows better circulation to the probe, less ambient light interference resulting in better readings & therefore better oxygen management.
